(19) United States
(12) Patent Application Publication
(10) Pub. No.: US 2014/0318553 A1
(43) Pub. Date: Oct. 30, 2014

(54) Soft Body Restraint

(71) Applicant: Dawn WOLFE, Sandy, UT (US)

(72) Inventor: Dawn WOLFE, Sandy, UT (US)

(21) Appl. No.: 13/874,415

(22) Filed: Apr. 30, 2013

(51) Int. Cl. A61F 5/37

(52) U.S. Cl. CPC A61F 5/3776
USPC 128/878

(57) ABSTRACT

A soft body restraint which can safely and comfortably restrain an individual. The soft body restraint having a soft folded material and an anchor mechanism. A usable section of the soft folded material is pulled through a first hole and/or a second hole of the soft folded sheet in order to form a first slipknot and/or second slipknot, respectively. An individual can insert their wrist(s) through the first slipknot and/or second slipknot. The user can then tighten the first slipknot and/or second slipknot by pulling the usable section in the opposite direction in order to bind their wrists together. The anchor mechanism can be used to secure the soft folded body to a desired object and has a variable length strap and a buckle. The variable length strap anchors the soft folded sheet to the desired object while the buckle allows the user to adjust the distance between the two.

### FIELD OF THE INVENTION

The present invention relates generally to a restraining apparatus. More specifically, the present invention is a soft body restraint that may be utilized to secure or restrain an individual without hurting or irritating the skin of the individual.

### BACKGROUND OF THE INVENTION

Handcuffs are a popular form of body restraint and have been around for many years. The primary use of handcuffs is to restrain an individual's hands together in order to prevent them from escaping. The handcuffs used by law enforcement are typically constructed from metal but can also be made from high grade plastics and composites. These handcuffs are designed for strength and durability. Handcuffs were initially intended to restrain outlaws and prisoners, however, their use in other situations has become increasingly popular. Outside of law enforcement, handcuffs are commonly used to playfully restrain someone. While metal handcuffs are perfect for retraining prisoners, they do not offer much comfort when being used in a playful manner. For this reason fuzzy handcuffs were produced. Fuzzy handcuffs are typically made from metal as well, however, they feature a soft layer of material that is wrapped around both wrist portions. While fuzzy handcuffs provide more comfort than traditional metal handcuffs, they can still result in injury from banging one's wrist into another object. Additionally, handcuffs in general can be dangerous when used playfully, as they can be difficult to unlock. Also, if the handcuff key is misplaced or lost, an individual may be undesirably restrained for an extended period of time.

Therefore it is the object of the present invention to provide a body restraint which safely and comfortably secures an individual. The present invention is made from a soft material, such that it will not irritate the skin of a user. No mechanical locking mechanisms are used, so the present invention can be safely removed at any time. This allows individuals to experience the use of restraints in a playful manner without worrying about becoming trapped and unable to escape.

### BRIEF DESCRIPTION OF THE DRAWINGS

FIG. 1 is a perspective view of the soft body restraint in the first embodiment of the present invention.

FIG. 2 is a top elevational view of the soft body restraint in the first embodiment of the present invention.

FIG. 3 is a right side elevational view of the soft body restraint in the first embodiment of the present invention.

FIG. 4 is a diagram depicting the use of the soft body restraint in the first embodiment of the present invention.

FIG. 5 is a perspective view of the top of the soft folded sheet and first grommet assembly in the first embodiment of the present invention.

FIG. 6 is a perspective view of the bottom of the soft folded sheet and first grommet assembly in the first embodiment of the present invention.

FIG. 7 is a front elevational view of the soft folded sheet and first grommet assembly in the first embodiment of the present invention.

FIG. 8 is a left side elevational view of the soft folded sheet and first grommet assembly in the first embodiment of the present invention.

FIG. 9 is a perspective view of the usable section of the soft folded sheet looped through the first grommet to form the first slipknot in the first embodiment of the present invention.

FIG. 10 is a perspective view of the soft body restraint in the second embodiment of the present invention.

FIG. 11 is a top elevational view of the soft body restraint in the second embodiment of the present invention.

FIG. 12 is a right side elevational view of the soft body restraint in the second embodiment of the present invention.

FIG. 13 is a diagram depicting the use of the soft body restraint in the second embodiment of the present invention.

FIG. 14 is a perspective view of the top of the soft folded sheet and first grommet assembly in the second embodiment of the present invention.

FIG. 15 is a perspective view of the bottom of the soft folded sheet and first grommet assembly in the second embodiment of the present invention.

FIG. 16 is a front elevational view of the soft folded sheet and first grommet assembly in the second embodiment of the present invention.

FIG. 17 is a left side elevational view of the soft folded sheet and first grommet assembly in the second embodiment of the present invention.

FIG. 18 is a perspective view of the usable section of the soft folded sheet looped through the first grommet to form the first slipknot in the second embodiment of the present invention.

### DETAIL DESCRIPTIONS OF THE INVENTION

All illustrations of the drawings are for the purpose of describing selected versions of the present invention and are not intended to limit the scope of the present invention.

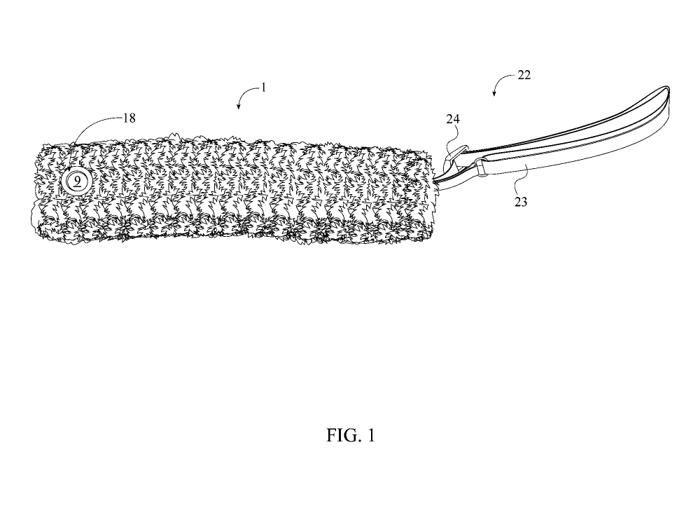

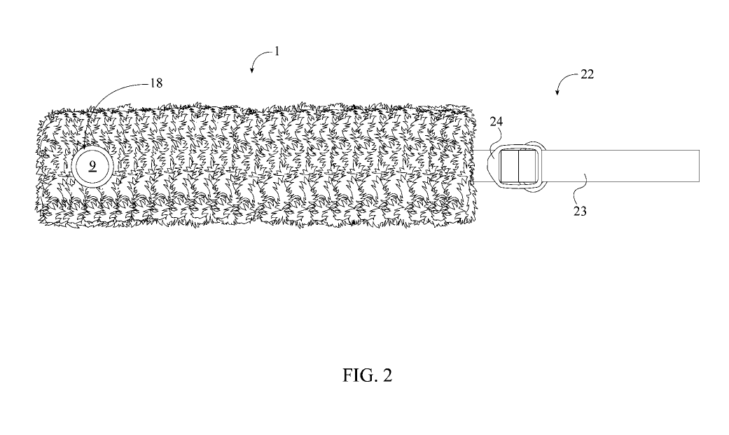

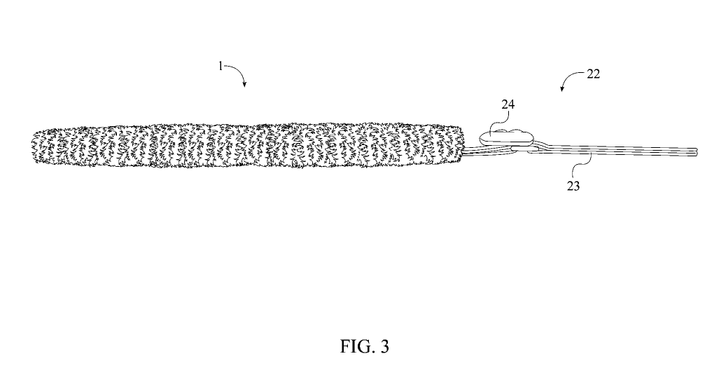

The present invention is a soft body restraint, which can be used to both safely and comfortably restrain an individual. The soft body restraint is typically used to bind an individual's hands together by being placed around the wrists, however, the soft body restraint may be used with any portion of the body. While the soft body restraint is used to restrain humans in the preferred embodiment of the present invention, it may also be used to restrain animals or other objects. In a first embodiment of the present invention, the soft body restraint comprises a soft folded sheet 1, a first grommet 18 and an anchor mechanism 22, as shown in FIG. 1-4. The soft folded sheet 1 is the portion of the soft body restraint that is placed around the individual's wrist, while the anchor mechanism 22 secures the soft body restraint to another object, such as a bed post or chair.

In reference to FIG. 1-4, the outer surface of the soft body restraint is a soft material, such as faux fur or silk, in order to prevent the individual's skin from becoming irritated. In the first embodiment, the soft folded sheet 1 comprises a top layer 2, a bottom layer 3, a fold edge 7, and a first hole 9. The fold edge 7 is positioned along both the top layer 2 and bottom layer 3 such that it hingedly connects the top layer 2 and bottom layer 3. The top layer 2 is folded over the bottom layer 3 along the fold edge 7 to create a two layer strip of material, thus providing additional padding for the individual's wrist. Additionally, this allows for the inner surface of the soft folded sheet 1 to be a rough material as it does not come in contact with the individual's skin.

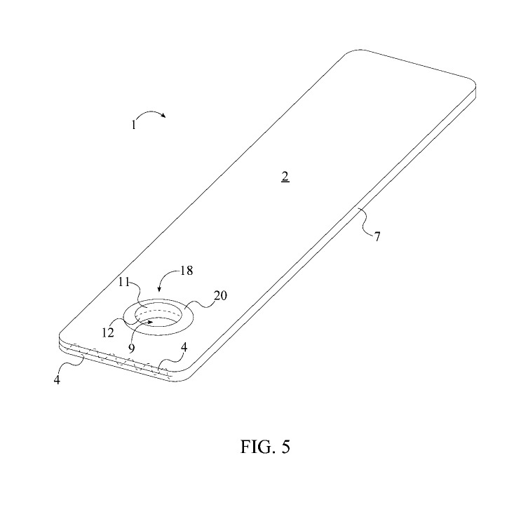

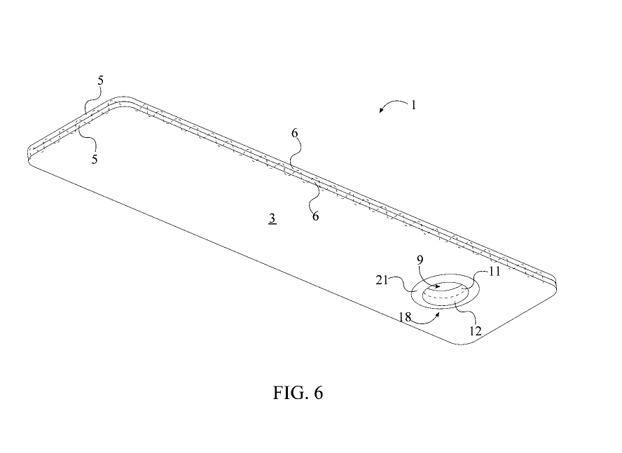

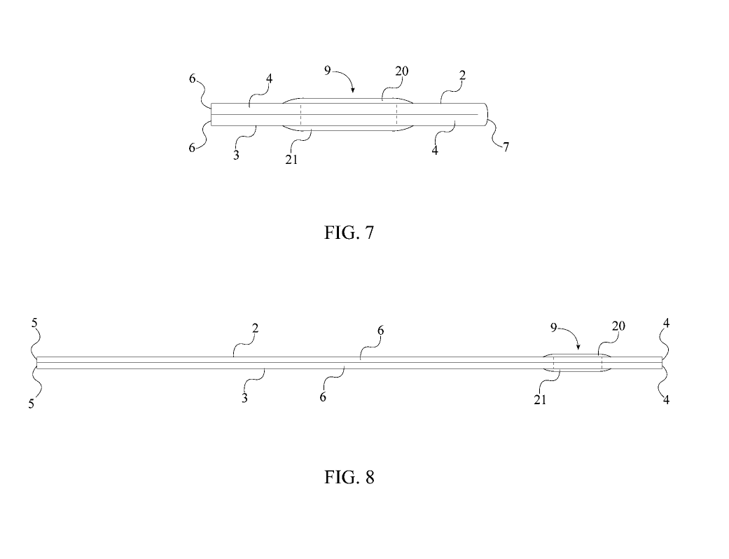

In reference to FIG. 5-8, the top layer 2 is connected to the bottom layer 3 to form a strip of material with two soft sides. The top layer 2 and the bottom layer 3 each comprise a first lateral edge 4, a second lateral edge 5, and a longitudinal edge 6. The first lateral edge 4, second lateral edge 5, and longitudinal edge 6 are used to align the top layer 2 with the bottom layer 3 in order to ensure that the top layer 2 and bottom layer 3 are properly connected. The first lateral edge 4 of the top layer 2 and the second lateral edge 5 of the top layer 2 are positioned opposite of each other along the top layer 2, while the longitudinal edge 6 of the top layer 2 is positioned opposite of the fold edge 7 across the top layer 2. The first lateral edge 4, second lateral edge 5, and longitudinal edge 6 of the top layer 2 and the fold edge 7 create the perimeter of the top layer 2. Similarly, the first lateral edge 4 of the bottom layer 3 and the second lateral edge 5 of the bottom layer 3 are positioned opposite of each other along the bottom layer 3, while the longitudinal edge 6 of the bottom layer 3 is positioned opposite of the fold edge 7 across the bottom layer 3. The first lateral edge 4, second lateral edge 5, and longitudinal edge 6 of the bottom layer 3 and the fold edge 7 create the perimeter of the bottom layer 3.

In reference to FIG. 5-8, the first lateral edge 4, second lateral edge 5, and longitudinal edge 6 of both the top layer 2 and bottom layer 3 are positioned in a particular manner in order to connect the top layer 2 to the bottom layer 3. The first lateral edge 4 of the bottom layer 3 is positioned adjacent to the first lateral edge 4 of the top layer 2, allowing the first lateral edge 4 of the bottom layer 3 to be connected to the first lateral edge 4 of the top layer 2. Likewise, the second lateral edge 5 of the bottom layer 3 is positioned adjacent to the second lateral edge 5 of the top layer 2, allowing the second lateral edge 5 of the bottom layer 3 to be connected to the second lateral edge 5 of the top layer 2. And, in the same manner, the longitudinal edge 6 of the bottom layer 3 is positioned adjacent to the longitudinal edge 6 of the top layer 2, allowing the longitudinal edge 6 of the bottom layer 3 to be connected to the longitudinal edge 6 of the top layer 2. In this way, a two layer strip of material with a soft outer surface is formed from the soft folded sheet 1. In one method of connection, the first lateral edge 4 of the bottom layer 3 is sewn to the first lateral edge 4 of the top layer 2; the second lateral edge 5 of the bottom layer 3 is sewn to the second lateral edge 5 of the top layer 2; and the longitudinal edge 6 of the bottom layer 3 is sewn to the longitudinal edge 6 of the top layer 2. This method of sewing allows stitching from a soft material, such as string or silk, to be utilized in order to create a durable connection without compromising the comfort of the soft body restraint.

In reference to FIG. 5-8, the first hole 9 traverses through both the top layer 2 and the bottom layer 3 of the soft folded sheet 1. The first hole 9 is positioned adjacent to the first lateral edge 4 of both the top layer 2 and the bottom layer 3 and comprises a top perimeter guide 11 and a bottom perimeter guide 12. The top perimeter guide 11 and the bottom perimeter guide 12 delineate the opening that is cut through the top layer 2 and bottom layer 3 to create the first hole 9. As such, the top perimeter guide 11 traverses through the top layer 2, while the bottom perimeter guide 12 traverses through the bottom layer 3. The top perimeter guide 11 and the bottom perimeter guide 12 are concentric such that the first hole 9 traverses straight through the soft folded sheet 1. The top perimeter guide 11 is connected to the bottom perimeter guide 12 to prevent tears from forming in the soft folded sheet 1 in the area surrounding the first hole 9. In the preferred method of connection, the first grommet 18 is used to connect the top perimeter guide 11 to the bottom perimeter guide 12. The first grommet 18 comprises a male fastener 20 and a female fastener 21, the male fastener 20 being positioned around the top perimeter guide 11 and the female fastener 21 being positioned around the bottom perimeter guide 12. The male fastener 20 engages the female fastener 21 to form a secure connection, allowing the first grommet 18 to reinforce the first hole 9. Ideally, the first grommet 18 is constructed from soft material, such as leather or rubber, which is strong and durable, yet comfortable.

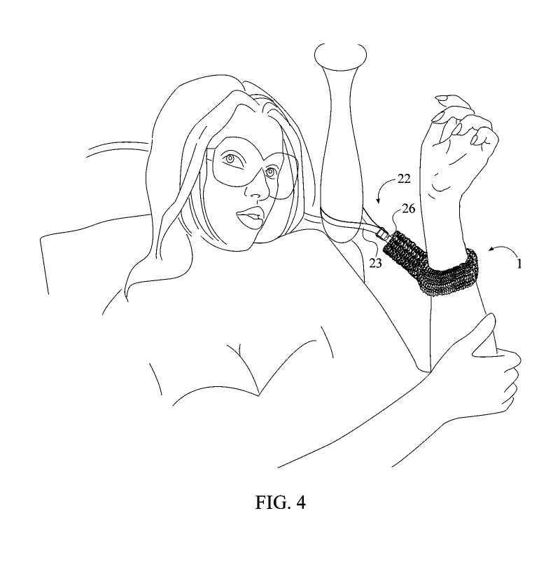

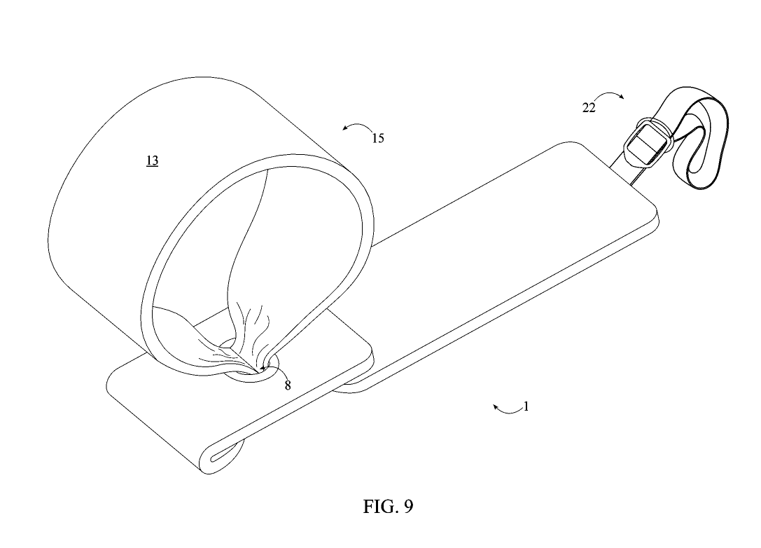

In reference to FIG. 4 and FIG. 9, the first hole 9 provides a channel in which a usable section 13 of the soft folded sheet 1 may be looped through in order to create a first slipknot 15. The usable section 13 surrounds the first hole 9 and is the portion of the soft folded sheet 1 that encircles the individual's wrist. A user takes a portion of the usable section 13 and pushes or pulls the portion through the first hole 9. As the usable portion is pushed or pulled through the first hole 9, the first slipknot 15 is formed by the looped portion of the soft folded sheet 1. The user can then continue to push or pull the usable section 13 through the first hole 9 until the first slipknot 15 is of the desired size. The individual can then position their wrist within the first slipknot 15 and pull the usable section 13 from the opposite side of the first hole 9 in order to tighten the first slipknot 15.

In reference to FIG. 1-4, while the soft folded sheet 1 is used to bind the individual's wrist, the anchor mechanism 22 secures the soft folded sheet 1 to another object, such as a bed post or chair. The anchor mechanism 22 comprises a variable length strap 23 and a buckle 24, which are used to determine the distance at which the soft folded sheet 1 is secured to the desired object. The variable length strap 23 is adjacently connected to the soft folded sheet 1 and is positioned opposite of the first hole 9 along the soft folded sheet 1. In this way, the anchor mechanism 22 does not interfere with the first slipknot 15. In the preferred method of connection, the variable length strap 23 is sewn to the soft folded sheet 1. The variable length strap 23 is looped around the desired object and holds the soft folded sheet 1 in place. The buckle 24 is positioned along the variable length strap 23 and slideably engages the variable length strap 23. The buckle 24 allows the user to change the length of the variable length strap 23 according to the current desired use.

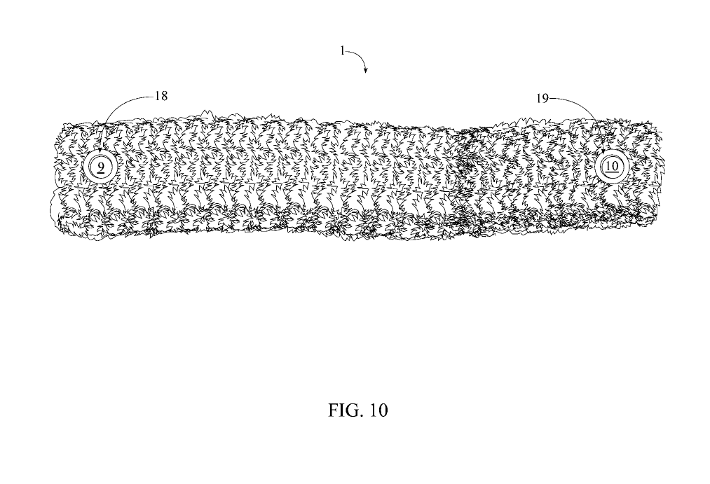

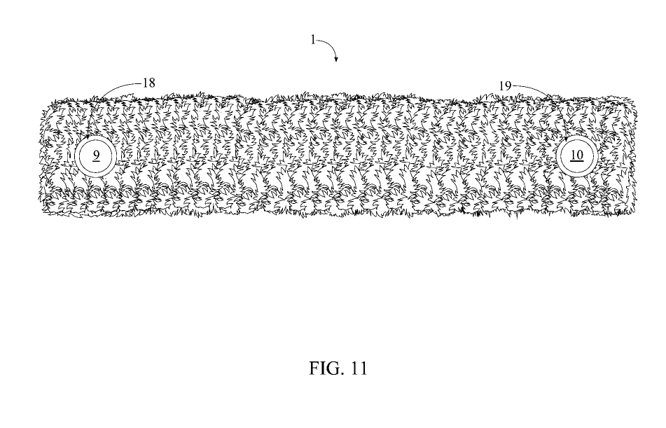

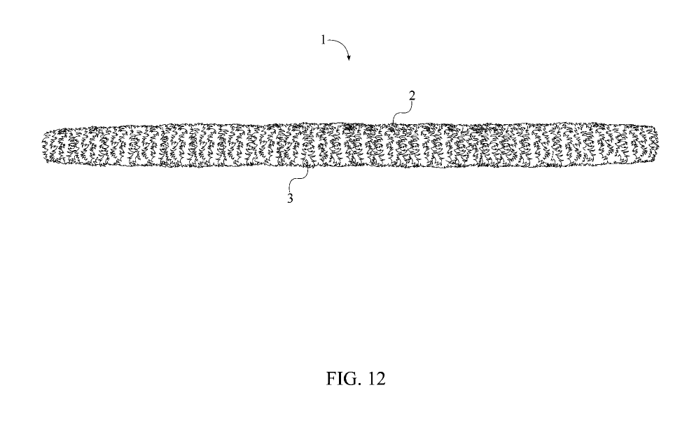

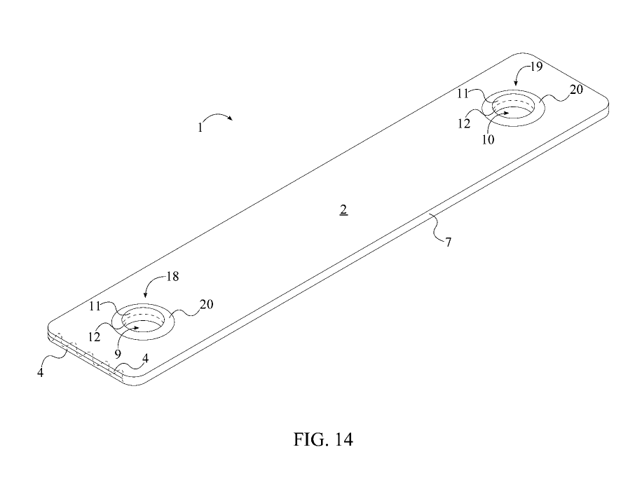

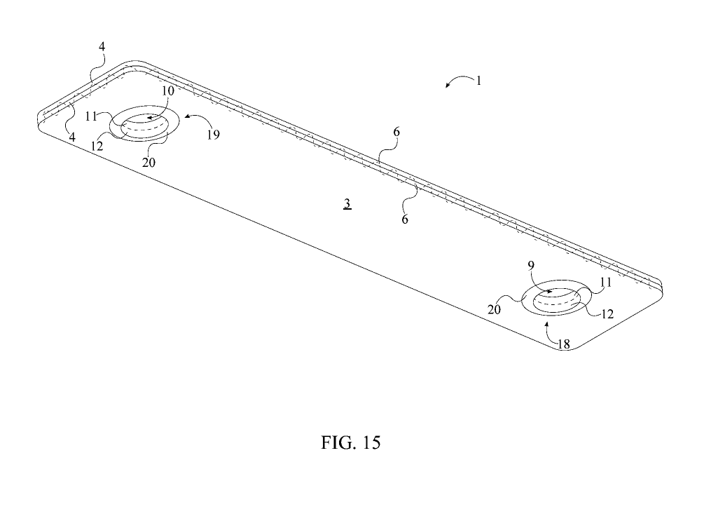

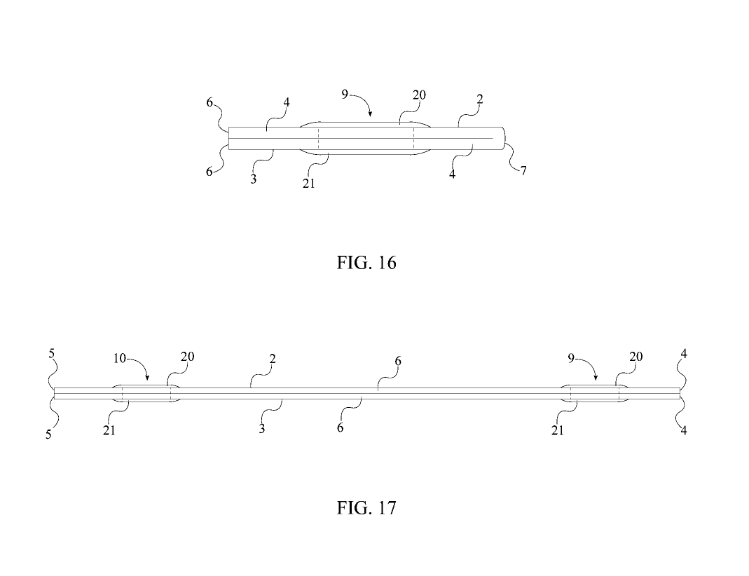

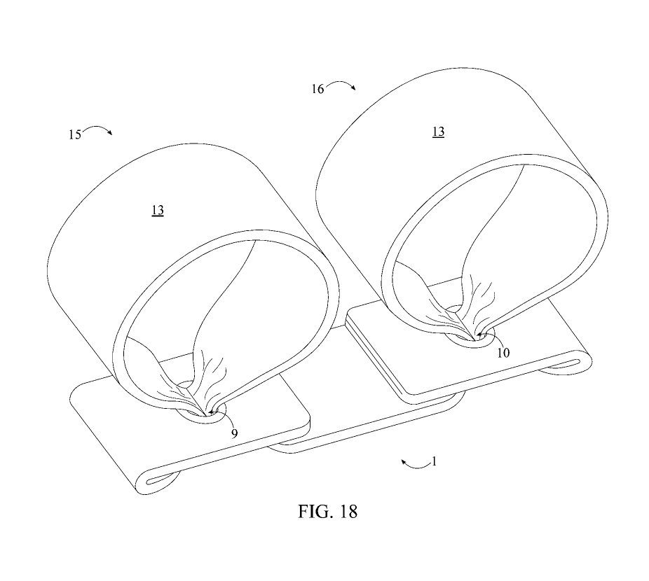

In reference to FIG. 10-12 and FIG. 14-17, in a second embodiment of the present invention, the soft body restraint does not comprise the anchor mechanism 22 but further comprises a second grommet 19. Additionally, the soft folded sheet 1 further comprises a second hole 10. Similar to the first hole 9, the second hole 10 traverses through both the top layer 2 and bottom layer 3 and comprises a top perimeter guide 11 and a bottom perimeter guide 12. Both the top perimeter guide 11 and the bottom perimeter guide 12 of the second hole 10 delineate the opening that is cut through the top layer 2 and bottom layer 3 to create the second hole 10. As such, the top perimeter guide 11 of the second hole 10 traverses through the top layer 2, while the bottom perimeter guide 12 of the second hole 10 traverses through the bottom layer 3. The top perimeter guide 11 of the second hole 10 and the bottom perimeter guide 12 of the second hole 10 are concentric such that the second hole 10 traverses straight through the soft folded sheet 1. The top perimeter guide 11 of the second hole 10 is connected to the bottom perimeter guide 12 of the second hole 10 to prevent tears from forming in the soft folded sheet 1 in the area surrounding the second hole 10. In the preferred method of connection, the second grommet 19 is used to connect the top perimeter guide 11 of the second hole 10 to the bottom perimeter guide 12 of the second hole 10. The second grommet 19 comprises a male fastener 20 and a female fastener 21. The male fastener 20 of the second grommet 19 is positioned around the top perimeter guide 11 of the second hole 10 and the female fastener 21 of the second grommet 19 is positioned around the bottom perimeter guide 12 of the second hole 10. The male fastener 20 of the second grommet 19 engages the female fastener 21 of the second grommet 19 to form a secure connection, allowing the second grommet 19 to reinforce the second hole 10.

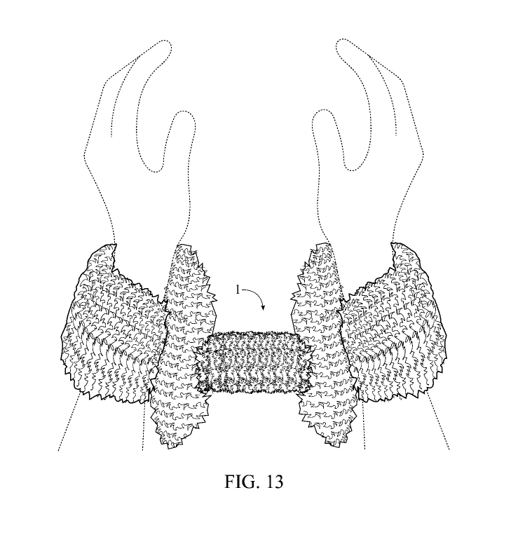

In reference to FIG. 13 and FIG. 18, in the second embodiment, the first hole 9 and the second hole 10 are positioned opposite of each other along the soft folded sheet 1. In this way, the first hole 9 provides the channel in which the usable section 13 may be looped through in order to create the first slipknot 15, while the second hole 10 provides a second channel in which the usable section 13 may be looped through in order to create a second slipknot 16. The individual can then position one wrist within the first slipknot 15 and the other wrist within the second slipknot 16, thus binding the individual's wrists together.

Although the invention has been explained in relation to its preferred embodiment, it is to be understood that many other possible modifications and variations can be made without departing from the spirit and scope of the invention as hereinafter claimed.

What is claimed is:

1. A soft body restraint comprises:
a soft folded sheet;
a first grommet;
an anchor mechanism;
the soft folded sheet comprises a top layer, a bottom layer, a fold edge, and a first hole;
the first grommet comprises a male fastener and a female fastener;
the anchor mechanism comprises a variable length strap and a buckle;
the top layer and the bottom layer each comprise a first lateral edge, a second lateral edge, and a longitudinal edge;
the first hole comprises a top perimeter guide and a bottom perimeter guide;
the fold edge being positioned along both the top layer and the bottom layer; and
the bottom layer being hingedly connected to the top layer by the fold edge.

2. The soft body restraint as claimed in claim 1 comprises:
the first lateral edge of the top layer and the second lateral edge of the top layer being positioned opposite of each other along the top layer;
the longitudinal edge of the top layer being positioned opposite of the fold edge across the top layer;
the first lateral edge of the bottom layer and the second lateral edge of the bottom layer being positioned opposite of each other along the bottom layer; and
the longitudinal edge of the bottom layer being positioned opposite of the fold edge across the bottom layer.

3. The soft body restraint as claimed in claim 1 comprises:
the first lateral edge of the bottom layer being positioned adjacent to the first lateral edge of the top layer;
the first lateral edge of the bottom layer being connected to the first lateral edge of the top layer;
the second lateral edge of the bottom layer being positioned adjacent to the second lateral edge of the top layer;
the second lateral edge of the bottom layer being connected to the second lateral edge of the top layer;
the longitudinal edge of the bottom layer being positioned adjacent to the longitudinal edge of the top layer; and
the longitudinal edge of the bottom layer being connected to the longitudinal edge of the top layer.

4. The soft body restraint as claimed in claim 3 comprises:
the first lateral edge of the top layer being sewn to the first lateral edge of the bottom layer;
the second lateral edge of the top layer being sewn to the second lateral edge of the bottom layer; and
the longitudinal edge of the top layer being sewn to the longitudinal edge of the bottom layer.

5. The soft body restraint as claimed in claim 1 comprises:
the first hole traversing through both the top layer and the bottom layer;
the first hole being positioned adjacent to the first lateral edge of both the top layer and the bottom layer;
the top perimeter guide traversing through the top layer;
the bottom perimeter guide traversing through the bottom layer;
the top perimeter guide and the bottom perimeter guide being concentric to each other; and
the bottom perimeter guide being connected to the top perimeter guide.

6. The soft body restraint as claimed in claim 5 comprises:
the male fastener being positioned around the top perimeter guide;
the female fastener being positioned around the bottom perimeter guide; and
the female fastener being engaged by the male fastener.

7. The soft body restraint as claimed in claim 1 comprises:
the variable length strap being adjacently connected to the soft folded sheet;
the variable length strap being positioned opposite of the hole along the soft folded sheet;
the buckle being positioned along the variable length strap; and
the buckle slideably engaging the variable length strap.

8. The soft body restraint as claimed in claim 7 comprises:
the variable length strap being sewn to the soft folded sheet.

9. The soft body restraint as claimed in claim 1 comprises:
the soft folded sheet further comprises a usable section;
the usable section surrounding the first hole; and
the usable section being looped through the hole in order to create a first slipknot.

10. A soft body restraint comprises:
a soft folded sheet;
a first grommet;
a second grommet;
the soft folded sheet comprises a top layer, a bottom layer, a fold edge, a first hole, and a second hole;
the first grommet and the second grommet each comprise a male fastener and a female fastener;
the top layer and the bottom layer each comprise a first lateral edge, a second lateral edge, and a longitudinal edge;
the first hole and the second hole each comprise a top perimeter guide and a bottom perimeter guide;
the fold edge being positioned along both the top layer and the bottom layer; and
the bottom layer being hingedly connected to the top layer by the fold edge.

11. The soft body restraint as claimed in claim 10 comprises:
the first lateral edge of the top layer and the second lateral edge of the top layer being positioned opposite of each other along the top layer;
the longitudinal edge of the top layer being positioned opposite of the fold edge across the top layer;
the first lateral edge of the bottom layer and the second lateral edge of the bottom layer being positioned opposite of each other along the bottom layer; and
the longitudinal edge of the bottom layer being positioned opposite of the fold edge across the bottom layer.

12. The soft body restraint as claimed in claim 10 comprises:
the first lateral edge of the bottom layer being positioned adjacent to the first lateral edge of the top layer;
the first lateral edge of the bottom layer being connected to the first lateral edge of the top layer;
the second lateral edge of the bottom layer being positioned adjacent to the second lateral edge of the top layer;
the second lateral edge of the bottom layer being connected to the second lateral edge of the top layer;
the longitudinal edge of the bottom layer being positioned adjacent to the longitudinal edge of the top layer; and
the longitudinal edge of the bottom layer being connected to the longitudinal edge of the top layer.

13. The soft body restraint as claimed in claim 12 comprises:
the first lateral edge of the top layer being sewn to the first lateral edge of the bottom layer;
the second lateral edge of the top layer being sewn to the second lateral edge of the bottom layer; and
the longitudinal edge of the top layer being sewn to the longitudinal edge of the bottom layer.

14. The soft body restraint as claimed in claim 10 comprises:
the first hole and the second hole traversing through both the top layer and the bottom layer;
the first hole and the second hole being positioned opposite of each other along the soft folded sheet;
the top perimeter guide traversing through the top layer;
the bottom perimeter guide traversing through the bottom layer;
the top perimeter guide and the bottom perimeter guide being concentric to each other; and
the bottom perimeter guide being connected to the top perimeter guide.

15. The soft body restraint as claimed in claim 14 comprises:
the male fastener being positioned around the top perimeter guide;
the female fastener being positioned around the bottom perimeter guide; and
the female fastener being engaged by the male fastener.

16. The soft body restraint as claimed in claim 10 comprises:
the soft folded sheet further comprises a usable section;
the usable section surrounding the first hole and the second hole;
the usable section being looped through the first hole in order to create a first slipknot; and
the usable section being looped through the second hole in order to create a second slipknot.

17. A soft body restraint comprises:
a soft folded sheet;
a grommet;
an anchor mechanism;
the soft folded sheet comprises a top layer, a bottom layer, a fold edge, and a hole;
the grommet comprises a male fastener and a female fastener;
the anchor mechanism comprises a variable length strap and a buckle;
the top layer and the bottom layer each comprise a first lateral edge, a second lateral edge, and a longitudinal edge;
the hole comprises a top perimeter guide and a bottom perimeter guide;
the fold edge being positioned along both the top layer and the bottom layer;
the bottom layer being hingedly connected to the top layer by the fold edge;
the first lateral edge of the top layer and the second lateral edge of the top layer being positioned opposite of each other along the top layer;
the longitudinal edge of the top layer being positioned opposite of the fold edge across the top layer;
the first lateral edge of the bottom layer and the second lateral edge of the bottom layer being positioned opposite of each other along the bottom layer;
the longitudinal edge of the bottom layer being positioned opposite of the fold edge across the bottom layer;
the hole traversing through both the top layer and the bottom layer;
the hole being positioned adjacent to the first lateral edge of both the top layer and the bottom layer;
the variable length strap being adjacently connected to the soft folded sheet; and
the variable length strap being positioned opposite of the hole along the soft folded sheet.

18. The soft body restraint as claimed in claim 17 comprises:
the first lateral edge of the bottom layer being positioned adjacent to the first lateral edge of the top layer;
the first lateral edge of the top layer being sewn to the first lateral edge of the bottom layer;
the second lateral edge of the bottom layer being positioned adjacent to the second lateral edge of the top layer;
the second lateral edge of the top layer being sewn to the second lateral edge of the bottom layer;
the longitudinal edge of the bottom layer being positioned adjacent to the longitudinal edge of the top layer;
the longitudinal edge of the top layer being sewn to the longitudinal edge of the bottom layer; and
the variable length strap being sewn to the soft folded sheet.

19. The soft body restraint as claimed in claim 17 comprises:
the top perimeter guide traversing through the top layer;
the bottom perimeter guide traversing through the bottom layer;
the top perimeter guide and the bottom perimeter guide being concentric to each other;
the bottom perimeter guide being connected to the top perimeter guide;
the male fastener being positioned around the top perimeter guide;
the female fastener being positioned around the bottom perimeter guide;
the female fastener being engaged by the male fastener;
the buckle being positioned along the variable length strap; and
the buckle slideably engaging the variable length strap.

20. The soft body restraint as claimed in claim 17 comprises:
the soft folded sheet further comprises a usable section;
the usable section surrounding the hole; and
the usable section being looped through the hole in order to create a slipknot.
